(19) United States
(12) Patent Application Publication
(10) Pub. No.: US 2026/0315234 A1
(43) Pub. Date: Oct. 15, 2026

(54) SYSTEM FOR SCREW LOCATION OPTIMIZATION IN ORTHOPEDIC IMPLANTS

(71) Applicant: Zimmer, Inc., Warsaw, IN (US)

(72) Inventors: Bradley Elliott, Fort Wayne, IN (US); Maged Awadalla, Warsaw, IN (US); Robbin Claire LeJeune Monceaux, South Whitley, IN (US); Niloufar Shekouhi, Warsaw, IN (US)

(21) Appl. No.: 19/420,014

(22) Filed: Dec. 15, 2025

Related U.S. Application Data
(60) Provisional application No. 63/746,732, filed on Jan. 17, 2025.

(51) Int. Cl. A61F 2/30; A61F 2/40

(52) U.S. Cl. CPC A61F 2/30942; A61F 2/4081; A61F 2002/30943; A61F 2002/30948; A61F 2002/30955; A61F 2002/4085

(57) ABSTRACT

A method for manufacturing an implant can include receiving, at a computing device, patient specific data related to a bone; generating an implant geometry of an implant based on the patient specific data; generating an initial screw trajectory and location for each of a plurality of peripheral screws for the implant and a central screw for the implant; determining, at the computing device, based on the initial screw trajectories and locations, an initial centroid of the plurality of peripheral screws and the central screw, and an initial stiffness ratio based on an initial stiffness of the plurality of peripheral screws and the central screw about a horizontal axis and an initial stiffness of the plurality of screws and the central screw about a vertical axis; and determining, by the computing device, an optimal screw trajectory and location for each of the plurality of peripheral screws and the central screw for the implant using finite element analysis and a machine learning model where the optimal screw trajectories and locations are chosen such that a final centroid of the plurality of peripheral screw locations and the central screw location provides for an altered reduced stiffness ratio from the initial stiffness ratio.

### CLAIM OF PRIORITY

This application claims the benefit of U.S. Provisional Patent Application Ser. No. 63/746,732, filed on Jan. 17, 2025, the benefit of priority of which is claimed hereby, and which is incorporated by reference herein in its entirety.

### TECHNICAL FIELD

The present application relates to an orthopedic system and specifically to a shoulder implant system.

### BACKGROUND

In a healthy shoulder, the proximal humerus is generally ball-shaped, and articulates within a socket formed by the scapula, called the glenoid, to form the shoulder joint. Some implant systems for the total replacement of the shoulder joint generally replicate the natural anatomy of the shoulder. Such implant systems can include a humeral component(stemmed or stemless), and an articulating head that articulates within the socket of a glenoid component implanted within the glenoid of the scapula. Reverse-type shoulder implant systems have been developed in which the conventional ball-and-socket configuration that replicates the natural anatomy of the shoulder is reversed, such that a concave recessed articulating component is provided at the proximal end of the humeral component that articulates against a convex portion of a glenosphere of a glenoid component implanted within the glenoid of the scapula.

However, mild to severe cases of glenoid bone loss often require an implant to specifically match the patient's anatomy. The Comprehensive® Vault Reconstruction System available from Zimmer Biomet Inc, of Warsaw, Indiana is designed using a CT scan of the patient's mild to severe bone defect. Case managers are then able to create patient-matched implants to fill bone voids using titanium implant with a porous coating which allows for biologic fixation strengthening the bone-implant construct.

The initial stability of the implant is often dependent on implant buildup (e.g. moment arm), press-fit (if present), patient's bone material property (e.g. density and Young's modulus), and the placement/length of screws that fixate the implant to the bone. The location and engagement length of the screws can have a significant impact on the implant's ability to resist forces and moments that may disrupt the stability and compromise fixation.

### OVERVIEW

To further illustrate apparatuses and methods disclosed herein, a non-limiting list of examples is provided here:

Example 1 is a method for manufacturing an implant including receiving, at a computing device, patient specific data related to a bone; generating an implant geometry of an implant based on the patient specific data; generating an initial screw trajectory and location for each of a plurality of peripheral screws for the implant and a central screw for the implant; determining, at the computing device, based on the initial screw trajectories and locations, an initial centroid of the plurality of peripheral screws and the central screw, and an initial stiffness ratio based on an initial stiffness of the plurality of peripheral screws and the central screw about a horizontal axis (e.g. superior inferior axis) and an initial stiffness of the plurality of screws and the central screw about a vertical axis (e.g. anterior posterior axis); and determining, by the computing device, an optimal screw trajectory and location for each of the plurality of peripheral screws and the central screw for the implant using finite element analysis and a machine learning model where the optimal screw trajectories and locations are chosen such that a final centroid of the plurality of peripheral screw locations and the central screw location provides for an altered stiffness ratio from the initial stiffness ratio.

Example 2 is the method of Example 1, wherein optionally determining the optimal screw trajectory and location for each of the plurality of peripheral screws and the central screw comprises: changing a portion of the implant data representing a location of the central screw from a first location to a second location; and simulating a stiffness of the implant in the horizontal axis and the vertical axis and determining a horizontal: vertical stiffness ratio.

Example 3 is the method of any of Examples 1-2, wherein optionally the altered stiffness ratio includes a reduced stiffness ratio and is at most a 2:1 ratio.

Example 4 is the method of any of Example 1-3, wherein optionally the altered stiffness ratio is chosen to be patient specific.

Example 5 is the method of any of Examples 1-4, wherein optionally determining the optimal screw trajectory and location for each of the plurality of peripheral screws and the central screw for the implant using material mapping, finite element analysis, and the machine learning model includes determining a need to offset the central screw.

Example 6 is the method of any of Examples 1-5, optionally further including determining an initial loading moment of a glenosphere on the implant (e.g. varying the glenosphere lateral and distal offset), and determining an optimal screw trajectory and location for each of the plurality of peripheral screws and the central screw for the implant using finite element analysis and the machine learning model includes determining a reduced final loading moment relative to the initial loading moment (e.g. reducing glenosphere lateral and distal offset).

Example 7 is the method of any of Examples 1-6, optionally further including simulating moving the glenosphere taper hole more distally and offsetting the central screw relative to the glenosphere taper of the implant to reduce the loading moment and micromotion.

Example 8 is the method of any of Examples 1-7, wherein optionally determining the optimal screw trajectory and location for each of the plurality of peripheral screws and the central screw for the implant using material mapping, finite element analysis, and the machine learning model includes simulating a plurality of different screw locations and their associated implant construct stiffness centroid.

Example 9 is the method of any of Examples 1-7, wherein optionally receiving the patient specific data comprises: receiving a scan of a patient's anatomy; and extracting design constraints for the implant from the scan, wherein the patient specific data is from a CT scan.

Example 10 is the method of any of Example 1-7, optionally further including generating an initial screw length for each of the plurality of peripheral screws and the central screw, and determining, by the computing device, an optimal screw trajectory, length, and location for each of the plurality of peripheral screws and the central screw for the implant using material mapping, finite element analysis, and the machine learning model.

Example 11 is the method of any of Examples 1-10, optionally further including receiving, at the computing device, a bone material density distribution of the bone, wherein determining the optimal screw trajectory, length, and location for each of the plurality of peripheral screws and the central screw for the implant using finite element analysis and the machine learning model includes taking into account the bone material distribution.

Example 12 is the method of any of Examples 1-11, wherein optionally determining the optimal screw trajectory, length, and location for each of the plurality of peripheral screws and the central screw includes accounting for surgeon variables in the finite element analysis and the machine learning model and includes taking into account the bone material distribution.

Example 13 is the method of any of Examples 1-12, wherein optionally one or more of the surgeon variables include glenosphere size, glenosphere orientation, screw torque, screw lengths, and inclination.

Example 14 is the method of any of Examples 1-13, wherein optionally the finite element analysis and machine learning model includes generating a plurality of designs for the implant, each of the plurality of designs including a different permutation of design parameters including screw trajectory and location for each of a plurality of peripheral screws for the implant and the central screw for the implant.

Example 15 is the method of any of Examples 1-14, optionally further including use all inputs and outputs of the method to build further ML models which can include patient long term outcomes.

Example 16 includes a system for manufacturing an implant, the system comprising: at least one processor; and at least one memory storing instructions that, when executed by the at least one processor, cause the at least one processor to perform actions comprising: receiving, at a computing device, patient specific data related to a bone; generating an implant geometry of an implant based on the patient specific data; generating an initial screw trajectory and location for each of a plurality of peripheral screws for the implant and a central screw for the implant; determining, at the computing device, based on the initial screw trajectories and locations, an initial centroid of the plurality of peripheral screws and the central screw, and an initial stiffness ratio based on an initial stiffness of the plurality of peripheral screws and the central screw about a horizontal axis (e.g. superior inferior) and an initial stiffness of the plurality of screws and the central screw about a vertical axis (e.g. anterior posterior); and determining, by the computing device, an optimal screw trajectory and location for each of the plurality of peripheral screws and the central screw for the implant using finite element analysis and a machine learning model where the optimal screw trajectories and locations are chosen such that a final centroid of the plurality of peripheral screw locations and the central screw location provides for an altered stiffness ratio from the initial stiffness ratio.

Example 17 is the system of Example 16, optionally further comprising generating a plurality of designs for the implant, each of the plurality of designs including a different permutation of screw trajectory and location for each of the plurality of peripheral screws for the implant and the central screw.

Example 18 is the system of any of Examples 16-17, wherein optionally determining the optimal screw trajectory and location for each of the plurality of peripheral screws and the central screw comprises: changing a portion of the implant data representing a location of the central screw from a first location to a second location (e.g. offsetting the central screw away from the glenosphere taper location); and simulating a stiffness of the implant in the horizontal axis and the vertical axis and determining a stiffness ratio.

Example 19 is the system of any of Examples 16-18, wherein optionally the altered stiffness ratio is determined based on a specific patient need.

Example 20 is glenoid baseplate including a baseplate body formed according to a PMI (patient-matched implant); and the baseplate body including a plurality of mounting holes extending though the baseplate body including two or more peripheral holes and a central hole; wherein locations and trajectories of the plurality of mounting holes on the baseplate is chosen according to a method comprising: generating an initial screw trajectory, location, and length for each of a plurality of peripheral screws for the implant and a central screw for the implant; determining, at a computing device, based on the initial screw trajectories and locations, an initial centroid of the plurality of peripheral screws and the central screw, and an initial stiffness ratio based on an initial stiffness of the plurality of peripheral screws and the central screw in a horizontal axis and an initial stiffness of the plurality of screws and the central screw in a vertical axis; and determining, by the computing device, an optimal screw trajectory and location for each of the plurality of peripheral screws and the central screw for the implant using finite element analysis and a machine learning model where the optimal screw trajectories and locations are chosen such that a final centroid of the plurality of peripheral screw locations and the central screw location provides for a reduced stiffness ratio from the initial stiffness ratio.

In Example 21, the apparatuses or methods of any one or any combination of Examples 1-20 can optionally be configured such that all elements or combinations of elements or options recited are available to use or select from.

These and other examples and features of the present apparatuses and methods will be set forth in part in the following Detailed Description. This Overview is intended to provide non-limiting examples of the present subject matter—it is not intended to provide an exclusive or exhaustive explanation. The Detailed Description below is included to provide further information about the present apparatus, systems, and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an arthroplasty system for a reverse shoulder implant, in accordance with one embodiment.

FIG. 2 shows a side view of a PMI (patient matched implant) baseplate in a patient's scapula, in accordance with one embodiment.

FIG. 3 shows a lateral view of the implant in the scapula, in accordance with one embodiment.

FIG. 4 shows a schematic representation of a baseplate, in accordance with one embodiment.

FIG. 5 shows a schematic representation of a second glenoid baseplate, in accordance with one embodiment.

FIG. 6 illustrates a method for manufacturing an implant, in accordance with one embodiment.

FIG. 7 shows a method of forming a PMI baseplate, in accordance with one embodiment

FIG. 8 illustrates an example schematic of computing device, in accordance with one embodiment.

### DETAILED DESCRIPTION

As used herein, the following directional definitions apply. Anterior and posterior mean nearer the front or nearer the rear of the body, respectively, proximal, and distal mean nearer to or further from the root of a structure, respectively, and medial and lateral mean nearer the sagittal plane or further from the sagittal plane, respectively. The sagittal plane is an imaginary vertical plane through the middle of the body that divides the body into right and left halves.

The present disclosure relates to design processes incorporating machine simulations or modeling to manufacture patient-specific shoulder implants. The design process can optimize implant parameters to reduce likelihood of post-operative complications.

Patient bone and implant data are input into a data processing software, finite element analysis, and machine learning model. The model simulates loading conditions and bone-implant interface to optimize design parameters like mounting screw trajectories, lengths, and locations.

The current alternative is manual placement of screws based on other design features of the implant or based on some physiological criteria, such as placement in the best quality bone.

The present system provides additional information about the effect of screw trajectories, locations, and length and their likely effect on transmission of forces and moments. This allows the user to evaluate various configurations in order to determine the most optimal placement for the patient or anatomy.

The present system provides a tool for implant designers and surgeons to assist with placement of screws in locations that provide optimal resistance to those forces and moments in order to limit disruption to the stability of the implant.

Specifically, the present system includes a software tool that allows the user to specify the initial location of implant screws, and then provide feedback to the user regarding the location of the screw pattern centroid. The user is then able to interactively reposition the screws in order to optimize the locations of the screws based on some desired centroid location. By knowing the location of the centroid for a given screw pattern, the user may be able to optimize placement of other features, such as tapers or other fixation hardware. Inversely, the user could optimize screw placement based on a mandatory fixed location of the centroid, such as in a case where loading must pass through a fixed taper.

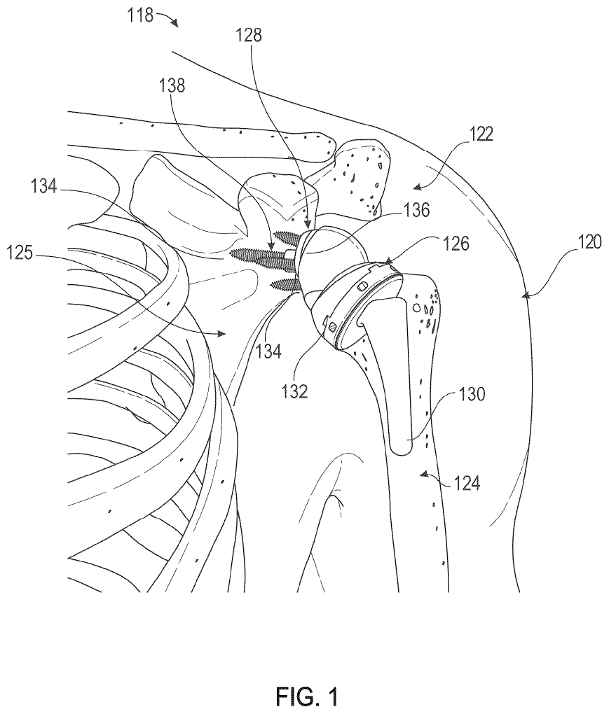

FIG. 1 shows a reverse shoulder implant, in accordance with one embodiment.

FIG. 1 shows that a surgical site 118 of a patient 120 can include, or can be located proximally to, a joint 122 including a humerus 124 and a scapula 125. In this example, the joint 122 can be a shoulder joint (e.g., the glenohumeral joint). The joint 122 can be at least partially replaced with a first implant 126 and a second implant 128.

The first implant 126 can be generally representative of a stemmed humeral implant adapted for use in a reverse shoulder replacement procedure. For example, the first implant 126 can include a stem 130 and an articulation component 132. The articulation component 132 can be, for example, a prosthetic humeral bearing securable to the stem 130 for articulating against a prosthetic humeral or glenosphere. The second implant 128 can be generally representative of a glenoid implant adapted for use in a reverse shoulder replacement procedure. For example, the second implant 128 can include a baseplate 134, a glenosphere 136, and a plurality of fixation components 138. The baseplate 134 can be representative of a variety of bases or baseplates adapted for fixation to bone within a glenoid cavity 148 of the scapula 125 of the patient 120.

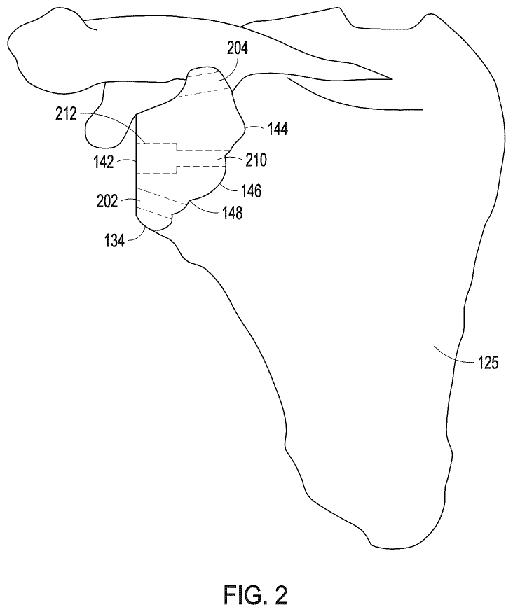

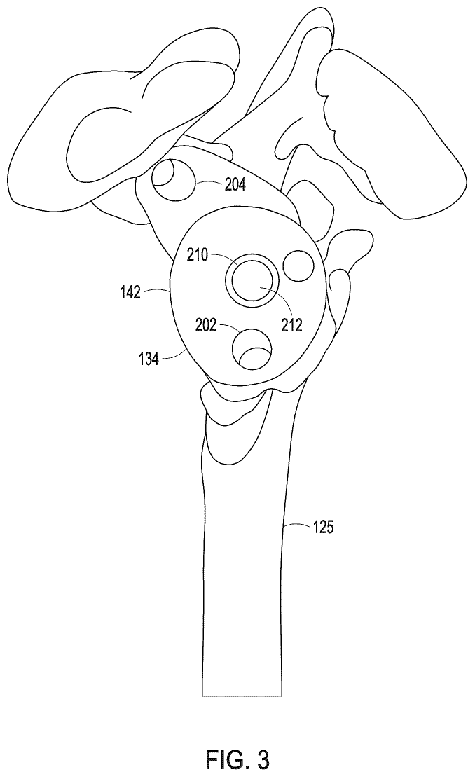

FIG. 2 shows a side view of a PMI (patient match implant) baseplate 134 implanted in a patient's scapula, in accordance with one embodiment, and FIG. 3 shows a lateral view of the baseplate 134 in the scapula 125, in accordance with one embodiment.

In some examples, such as in shoulder arthroplasties involving patients with severely deficient rotator cuffs or extensive scapular bone loss, the baseplate 134 can be an augmented patient-specific glenoid implant. For example, the baseplate 134 can include an exterior portion defining an outer contacting surface 144 that is patient-specific. For example, the outer contacting surface 144 can be sized and shaped to conform to, and make substantial surface contact with, a glenoid surface 146 defined by the scapula 125. The glenoid surface 146 can represent one or more surfaces located within, or defining, a glenoid cavity 148 of the scapula 125.

In such examples, the exterior portion and the outer contacting surface 144 can each be sized and shaped based on a three-dimensional image or model of a shoulder joint of a patient, such as the joint 122 of the patient 120. In some examples, the baseplate 134 can be representative of a custom glenoid baseplate from the Comprehensive® Vault Reconstruction System available from Zimmer Biomet Inc, of Warsaw, Indiana. The baseplate 134 can also include a plurality of mounting holes 202, 204, 210 adapted to receive, and thereby guide, the plurality of fixation elements 138 (FIG. 1) into the scapula 125. The glenosphere 136 can be securable to the baseplate 134 for articulating against the articulation component 132 secured to the stem 130.

In preparation for a shoulder arthroplasty, various implant parameters of the baseplate 134 can first be optimized using the design process of the present disclosure, which is discussed in detail below.

During the shoulder arthroplasty, a surgeon can prepare the scapula 125 to receive the second implant 128. For example, the surgeon can ream, drill, or otherwise modify various bone surfaces within the glenoid cavity 148, such as the glenoid surface 146, to receive or otherwise contact and engage portions of the baseplate 134, or other portions of the second implant 128. After preparation of the scapula 125, the baseplate 134 can be affixed or secured thereto, such as by driving the plurality of fixation components 138 through the plurality of mounting holes into the scapula 125 using a surgical drill or screwdriver. The glenosphere 136 can then be secured to the baseplate 134, such as through a tapered or press-fit between one or more portions thereof.

Here, the glenoid baseplate implant 134 can include a baseplate body 142 formed according to a PMI design. Specifically, a back surface 144 of the implant 134 is shaped to conform to the specifics patient's glenoid cavity surface 146 of the glenoid cavity 148.

The baseplate 134 can include the plurality of mounting holes extending though the baseplate body 142 including two or more peripheral holes 202, 204 and a central hole 210. A glenosphere taper 212 is proved to mate with a taper of a glenosphere to be mounted to the baseplate 134. Although this example shows two peripheral mounting holes, other embodiments can include up to ten mounting holes around a periphery of the baseplate 134.

As noted above, the location, length, and trajectories of the fixation screws can have a significant impact on the implant's ability to resist forces and moments that may disrupt the stability and compromise fixation. There are many variables that can affect the locations and trajectories of the plurality of mounting holes 202, 204, 210 on the baseplate 134. The present system proved for guidance in choosing an optimal position for the mounting holes.

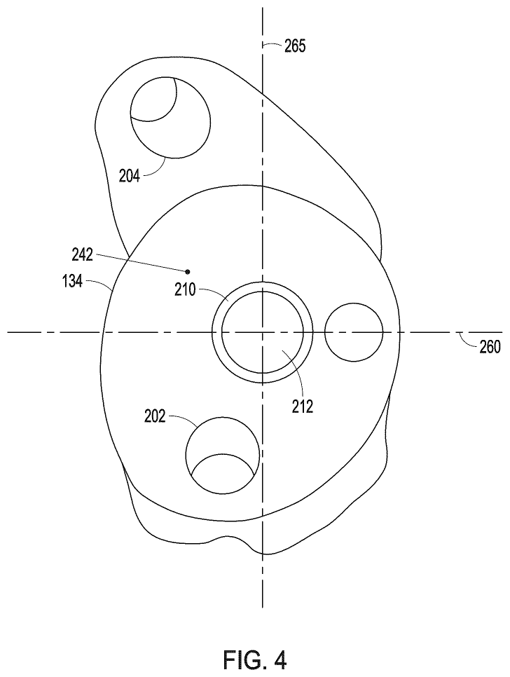

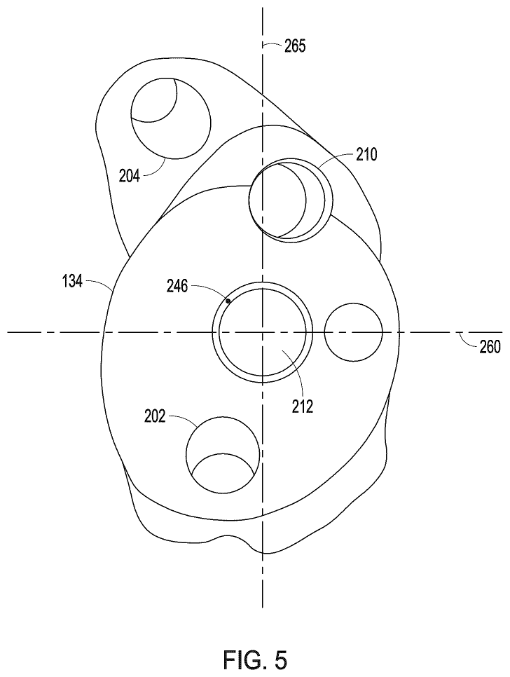

For example, FIG. 4 shows a representation of a baseplate 134 with a first set of mounting holes, in accordance with one embodiment, and FIG. 5 shows a representation of a second glenoid baseplate 134 having different mounting hole locations, in accordance with one embodiment.

Here, the baseplate 134, when implanted, defines a horizontal axis 260 (superior inferior axis), and a vertical axis 265 (anterior posterior axis). In FIG. 4, with the central hole 210 lined up with the taper hole 212, a centroid 242 of the mounting holes is shown. As will be further detailed below, with such a centroid the stiffness (resistance to bending) in the horizontal axis 260 is relatively much higher than the stiffness in the vertical axis 265 since the screws are nearer to the centerline. This situation can increase the micromotion (i.e. reduce the stability) of the implant.

In FIG. 5, with the central hole 210 moved away from the central glenosphere taper (off-set central screw) defining a new centroid 246, the stiffness ratio of the horizontal axis: vertical axis is much lower. For example, the stiffness ratio in FIG. 4 is approximately 10 or higher. In FIG. 5, the stiffness ratio can be 4:1, 3:1, 2:1 or lower or it can be patient specific based on patient anatomy and bone material property. One goal is to have the stiffness in each axis be equal to each other (i.e. a stiffness ratio of 1:1) or it can be patient specific allowing for the least amount of micromotion when implanted.

Moreover, other factors such as bone density and surgeon variables can also affect the stability of the implant. Accordingly, these other factors can also be taken into account along with the locations and trajectories of the central hole 210 and the plurality of peripheral holes. Moreover, other parameters of the implant including the moment arm of the glenosphere on the baseplate can be optimized, as will be discussed below.

Overall, the present system assists the designer with placement of the screws having an optimal length in locations and trajectories that provide optimal resistance to those forces and moments in order to limit disruption to the stability of the implant.

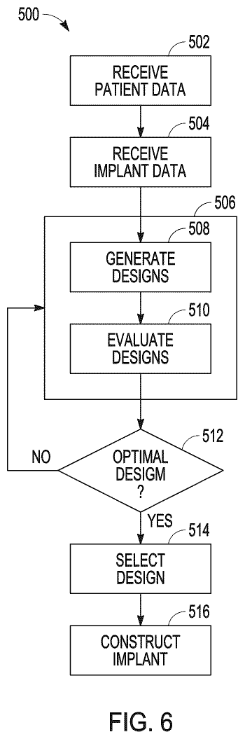

FIG. 6 illustrates a general method (500) for manufacturing an implant, in accordance with at least one example of this disclosure.

The method 500 can, in some examples, be generally representative of the design process as discussed with reference to the FIGs above. The steps or operations of the method 500 are illustrated in a particular order for convenience and clarity; many of the discussed operations can be performed by multiple different actors, devices, or systems. It is understood that subsets of the operations discussed in the method 500 can be attributable to a single actor, device, or system and can be considered a separate standalone process or method.

The method 500 can include a first stage 502. The first stage 502 can include receiving patient-specific data related to a bone. Receiving the patient-specific data can include receiving, at a computing device, one or more scans, such as including, but not limited to, X-rays, CT scans, MRI, fluoroscopic images, or other imaging data of a patient's anatomy relating to one or more bones of the patient, such as, a scapula of a shoulder joint of the patient.

The first stage 502 can also include extracting design constraints for an implant for the patient, such as, but not limited to, a baseplate of a shoulder implant. Next, using various image analysis techniques, physical characteristics or features of the patient's anatomy, such as, but not limited to, the bone density (based on pixel values that may represent bone density) of one or more bones of the patient, various geometric parameters of one or more bones of the patient (e.g., the dimensions of features or surfaces thereof), or the relative anatomical locations of bone, ligament, or tendons within the patient, can be determined or otherwise extracted. The bone density values, the relative anatomical locations, or the geometric parameters can be used as design constraints (e.g., implant parameters) for the implant for the patient, as they can be used to represent, for example, but not limited to, maximum geometric dimensions or constraints including a curvature, diameter, length, or otherwise a size and shape, for various surfaces or other features of the implant for the patient.

The method 500 can include a second stage 504. The second stage 504 can include receiving implant data related to an implant. Receiving the implant data can include determining maximum, minimum, and nominal values for design constraints (e.g., implant parameters) determined or otherwise extracted from the patient-specific imaging data related to the patient's anatomy during the first stage 502. For example, based on any of the bone density values, relative anatomical locations, or geometric parameters, determined or extracted from the patient-specific imaging data during the first stage 502, maximum, minimum, and nominal values for various geometrical dimensions, such as including, a curvature, diameter, length, or otherwise an overall size and shape, for various features or surfaces of the implant for the patient, can be determined.

The initial or nominal values can represent customary, or otherwise previously used, values that have been suitable or adequate for other patients having, or otherwise exhibiting, similar physical characteristics extracted or determined from imaging data. For example, if one or more previous patients exhibiting similar patient-specific imaging data were normally, or have otherwise been, fitted with an implant having X, Y, and Z implant parameters in the past, then the nominal values for the individual patient of the method 500 can be X, Y, and Z. In some examples, the second stage 504 can include receiving implant data from a database or repository for storing parameters of various stock or otherwise standardized implants routinely manufactured by an implant manufacturer. In such examples, the stock or standardized implants can represent the nominal values for various implant designs. In some examples, the second stage 504 can further include entering data, such as entered by a surgeon or other medical staff, for the implant for the patient based on a review of the patient-specific imaging data obtained during the first stage 502 and personal experience treating patients having similar imaging or scan data.

In some examples, the second stage 504 can include receiving implant parameters for the initial locations and trajectories of the peripheral mounting holes and the central hole of a glenoid implant, and initial screw lengths.

The method 500 can include a third stage 506. The third stage 506 can include determining an optimal implant design using the patient-specific data related to the bone and the implant data related to the implant. Determining an optimal implant design can include a first step 508 and a second step 510. The first step 508 can include generating one or more designs for the implant for the patient, such as, but not limited to, one or more design options for a baseplate of the implant. Generating the plurality of designs can include passing, as inputs, various patient related parameters, such as determined or extracted from the patient-specific imaging data during the first stage 502, such as, among others, bones density values, relative anatomical locations, and geometric parameters into a machine learning (hereinafter “ML”) model (e.g., a machine learning artificial intelligence model), and receiving or otherwise obtaining implant parameters as outputs from the ML model. In some examples, the generating the one or more designs can include generating a plurality of different design options by generating a number of different implant designs, using the ML model, that have different permutations of various implant parameters.

In some examples, the first step 508 can include selecting one or more standardized or standard line implant designs, such as, but not limited to, one or more standard mounting hole trajectory and location and one or more screw length.

The second step 510 can include evaluating the designs for the implant for the patient (e.g., the one or more design options or the plurality of design options of step 508). Evaluating the designs can include performing finite element analysis (hereinafter “FEA”) on each of the one or more design options for the implant. For example, during the FEA, a fit condition between the bone of the patient (e.g., the bone to receive or engage with the implant) can be simulated. Also, the influence of stiffness of the implant construct in the horizontal axis and vertical axis on micromotion and bone strain/strain energy can be analyzed.

Subsequently, in some examples, implant data representing any of various portions, segments, or areas of the implant, such as mounting hole locations and trajectories, and screw lengths can be changed from a first value to a second value, and the condition between the bone and the implant can be re-simulated, such as until a quality or suitable estimated implant design is achieved.

In further examples, the ratio of the stiffness about the horizontal axis vs. the stiffness about the vertical axis can also be determined or changed by performing simulations using the ML model. For example, one or more first values that represent mounting screw locations, lengths, and trajectories can be changed to one or more second values. Still further examples of implant parameters that can be changed from a first value to a second value, but are not limited to, one or more locations of the taper hole and surgeon variables. In such an example, such implant parameters can be changed from a first value to a second value, and the revised condition between the bone and the implant can be simulated using the ML model to determine if an improved implant design has been obtained.

In some examples, the second step 510 can also include determining regions of the bone of the patient with optimal stresses based on simulated loading of the implant. For example, during FEA modeling, various loads and/or moments may be placed on or applied to an implant design. In an example, loading is simulated with the implant virtually implanted in the target bone. Based on the loading and moments, stresses\strains within the bone can be determined. Areas where stresses\strains are above a predetermined level (bone stresses\strain in an intact bone) can be identified, and implant parameters of the baseplate can be changed to lower or minimize the stresses\strain.

The method 500 can include a fourth stage 512. The fourth stage 512 can include determining if the implant design is optimal for an individual patient. In some examples, an implant design can be considered optimal when the horizontal stiffness is equal to the vertical stiffness, the implant has reduced micromotion, and the moment arm is reduced. However, based on other factors, including modeled screw stresses within the bone and the loading arm moment on the taper hole, the optimal ratio of horizontal:vertical stiffness may be at most 2:1, 3:1, 4:1 or higher. In an example, the initial baseplate will have a stiffness ratio of about 10.5. The ratios and other factors can be iteratively compared and an optimal solution can be determined by the model. The fourth stage 512 can also include selecting an implant design, such as indicated by reference number 514. For example, once an optimal implant design is found, the optimal implant design can be selected for manufacturing.

The method 500 can include the fifth stage 516. The fifth stage 516 can include constructing the implant for the patient. Constructing the implant for the patient can include exporting the optimal design parameters for a selected optimal implant, such as determined during the third stage 506 and the fourth stage 512. For example, the optimal design parameters of the optimal implant design can be exported or otherwise transmitted via computing device to an automated manufacturing device such as, but not limited to, a three-dimensional printer, a computer numerical controlled (CNC) milling machine, or other devices or systems. In some examples, the implant for the patient can be constructed using additive manufacturing, such as built layer by layer from titanium or cobalt-chrome. In some examples, the fifth stage 516 can include post-processing, such as surface finishing or sterilization, to help ensure that the implant meets specifications and is safe for implantation, which may include fatigue testing or material characterization.

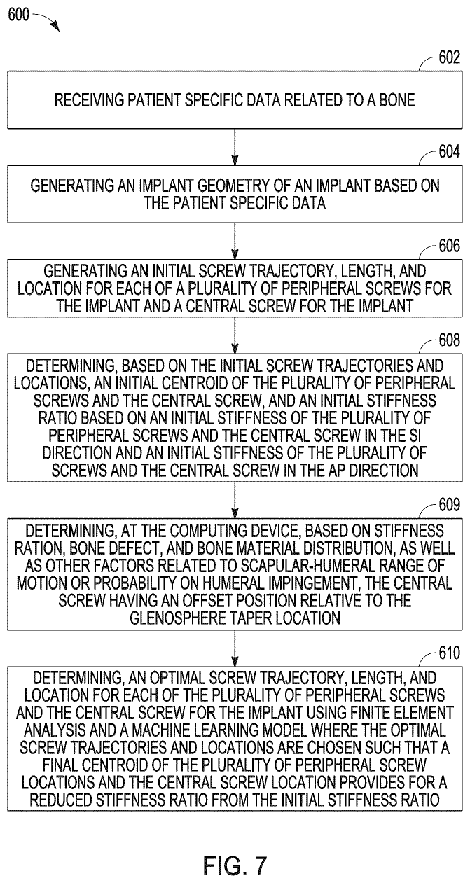

FIG. 7 shows a specific method of manufacturing an implant and choosing hole location and trajectories and screw lengths, in accordance with one embodiment.

Here, the method (600) includes receiving, at a computing device, patient specific data related to a bone (602). Specifically, a CT scan defining the geometry of a glenoid cavity in a scapula. wherein receiving the patient specific data can include receiving a scan of a patient's anatomy, and extracting design constraints for the implant from the scan, wherein the patient specific data is from a CT scan.

The method further includes generating an implant geometry of a glenoid implant based on the patient specific data such that the implant matches the defect (604).

The method further includes generating one or more of an initial screw trajectory, length, and location for each of a plurality of peripheral screws for the implant and a central screw for the implant (606).

The method further includes determining, at the computing device, based on the initial screw trajectories, lengths, and locations, an initial centroid of the plurality of peripheral screws and the central screw, and an initial stiffness ratio based on an initial stiffness of the plurality of peripheral screws and the central screw about the horizontal axis and an initial stiffness of the plurality of screws and the central screw about the vertical axis (608). As noted, in one example, the initial stiffness ratio may be about 10:1 ratio of the horizontal:vertical axis.

The method can further optionally include determining, at the computing device, based on stiffness ration, bone defect, and bone material distribution, as well as other factors related to scapular-humeral range of motion or probability on humeral impingement, the central screw having an offset position relative to the glenosphere taper location (609).

Then the method includes determining, by the computing device, one or more of an optimal screw trajectory, length, and location for each of the plurality of peripheral screws and the central screw for the implant using bone material mapping, finite element analysis, and a machine learning model (610), where the optimal screw trajectories, locations, and length are iteratively chosen such that a final centroid of the plurality of peripheral screw locations and the central screw location provides for an altered stiffness ratio from the initial stiffness ratio. As will be discussed below, this altered stiffness ratio can be a reduced stiffness ration and can be chosen to be as close to 1:1 as possible. In other examples, the altered stiffness ratio can be patient specific based on patient's bone anatomy and material property, but other design factors can be input into the machine learning model to require some compromises. For example, there can be instances where bone support is excellent in one direction, but not in the other. In situations like that, one option is to choose the stiffness based on the surrounding anatomy or other constraints.

The finite element analysis and machine learning model can iteratively generate a plurality of possible designs for the implant. Each of the plurality of possible designs can include a different permutation of design parameters including one or more of screw trajectory, length, and location for each of a plurality of peripheral screws for the implant and the central screw for the implant.

In some examples, determining the optimal screw trajectory and location for each of the plurality of peripheral screws and the central screw can include changing a portion of the implant data representing a location of the central screw from a first location to a second location and simulating a stiffness of the implant in the horizontal axis and vertical axis and determining a stiffness ratio.

In one example, the reduced stiffness ratio can be at most a 2:1 ratio of the stiffness of the plurality of screws in the horizontal axis compared to the stiffness of the plurality of screws in the vertical axis. As noted in some examples, the reduced stiffness ratio can be at most a 3:1 ratio of the stiffness of the plurality of screws in the horizontal axis compared to the stiffness of the plurality of screws in the vertical axis. In some examples, the reduced stiffness ratio can be at most a 4:1 ratio of the stiffness of the plurality of screws in the horizontal axis compared to the stiffness of the plurality of screws in the vertical axis. In one example, the selected design will have a stiffness is approximately equal in the horizontal axis and in the vertical axis. The stiffness ratio can also be patient specific based on patient bone anatomy and material property. The desired stiffness ratio can for each patient can be determined using a ML model based on retrospective cases, FEA evaluation and clinical outcomes.

Determining the optimal screw trajectory and location for each of the plurality of peripheral screws and the central screw for the implant using finite element analysis and a machine learning model can include determining a need to offset the central screw. For example, the central screw can be moved to reduce the stiffness ratio and to provide a centroid of all the mounting holes that is as close to the taper hole as possible. This will provide a lower moment arm on the baseplate from the glenosphere.

Accordingly, the method can further include determining an initial loading moment of a glenosphere on the implant, and determining an optimal screw trajectory, length, and location for each of the plurality of peripheral screws and the central screw for the implant using material mapping, finite element analysis and a machine learning model includes determining a reduced final loading moment relative to the initial loading moment. The method can further include simulating moving a taper hole of the implant to affect the loading moment. In some example, the method can include determining an opportunity to reduce the final loading but examining an optimized glenosphere size that can avoid impingement while reducing moment arm.

As noted, other variables can be input in the finite element analysis and machine learning model to determine design having an optimal screw length, location, and trajectory.

For example, the method can further include receiving, at the computing device, a bone material density distribution of the scapula. Determining the optimal screw trajectory, length, and location for each of the plurality of peripheral screws and the central screw for the implant using finite element analysis and the machine learning model includes can take into account the bone material distribution. The goal is for each screw to be of a length and location to be implanted in the highest density bone possible to reduce micromotion or examining other strategies for low density bone to reduce micromotion. Accordingly, in some designs, the optimal locations for screw location from a stiffness ratio standpoint may not be ideal when taking the bone density into account.

In another example, determining the optimal screw trajectory, length, and location for each of the plurality of peripheral screws and the central screw includes accounting for surgeon variables in the finite element analysis and the machine learning model. Surgeon variables are not always known, but if the information is available, the one or more of the surgeon variables can include glenosphere size, glenosphere orientation, screw torque, screw lengths, construct version, and inclination. If these variables are known, they can affect the moment arm of forces applied to the implant based on location of the taper hole of the implant.

As discussed above in FIG. 6, the method can also include exporting, by the computing device, the optimal screw trajectory, location, and length for each of the plurality of peripheral screws and the central screw. For example, exporting the optimal screw trajectory, length, and location for each of the plurality of peripheral screws and the central screw includes transmitting the screw trajectory, length, and location for each of the plurality of peripheral screws and the central screw to an automated manufacturing device.

Over time, the system can also use all the inputs and outputs of the methods above to build further ML models which can include patient long term outcomes.

Thus a general summary of the process can include:
1. Automatically generate a glenoid implant geometry based on surgeon's input.
2. Generate an initial screw trajectory, length, and location.
3. Calculate initial centroid and stiffness in the horizontal axis and the vertical axis.
4. Obtain bone material distribution from the CT.
5. Account for surgeon variabilities (implant buildup, glenosphere size and orientation, screw torque, and construct version and inclination).
6. Using an ML model powered with finite element analysis to analyze a relationship between patient/surgeon variability, and stability) in order to:
   A. Optimize screw location and trajectory.
   B. Determine the need to offset central screw.
   C. Find optimal screw length for stability (reduced micromotion).
7. Find the new centroid which provides reduced (or equal) implant construct stiffness in the horizontal axis and the vertical axis with reduced micromotion.
8. Send new design to surgeons.
9. Use all study inputs and output to build ML models which can include patient long term outcomes.

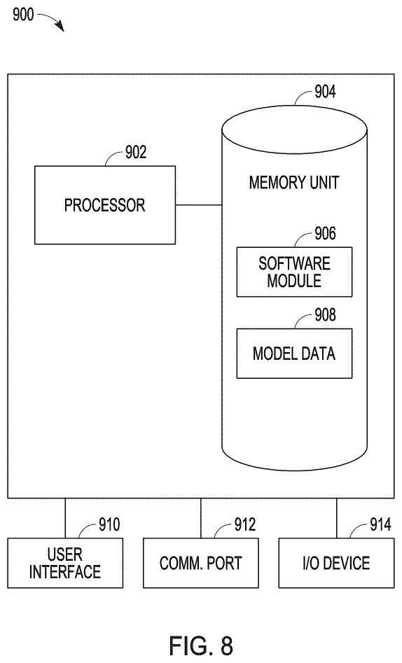

FIG. 8 illustrates an example schematic of computing device 900, in accordance with at least one example of this disclosure. As shown in FIG. 8, the computing device 900 can include a processor 902 and a memory unit 904. The memory unit 904 can include a software module 906 and model data 908. While executing on the processor 902, the software module 906 can perform processes for generating ML models, such as, but not limited to, generating one or more prosthetic implant designs, performing FEA modeling on one or more generated designs, which can include, for example, one or more stages of the method 500, or one or more stages of the method 600, described above with respect to FIG. 6 and FIG. 7 above, respectively. The model data 908 can include, but is not limited to, training data, design parameters used as inputs, databases, such as KNN databases, models generated using various prosthetic implant designs, or other data or information. The computing device 900 can also include a user interface 910, a communications port 912, and an input/output (I/O) device 914.

The user interface 910 can include any number of devices that allow a user to interface with the computing device 900. Some non-limiting examples of the user interface 910 can, but are not limited to, a keypad, a microphone, or a display (e.g., a touchscreen or otherwise). The communications port 912 can allow the computing device 900 to communicate with various information sources and devices, such as, but not limited to, remote computing devices, such as servers or other remote computers. For example, such remote computing devices can maintain data, such as model data, that can be retrieved by the computing device 900 using the communications port 912. Some non-limiting examples of the communications port 912 can include, but are not limited to, ethernet cards (e.g., wireless or wired), BLUETOOTH® transmitters and receivers, or near-field communications modules. The I/O device 914 can allow the computing device 900 to receive and output information. Some non-limiting examples of the I/O device 914 can include a camera (e.g., still camera or a video camera), or fingerprint or other biometric scanners. For example, the I/O device 914 can allow the computing device 900 to directly receive patient data from a CT scanning device, an X-ray machine, or other imaging devices.

In view of all the above, the design processes of the present disclosure, such as including the method 500 (FIG. 6) and the method 600 (FIG. 7) can be implemented to design a patient specific implant or generic or standardized line of patient implants including screw locations, lengths, and trajectories, where the ML models can identify the best or otherwise optimal implant design for a given or individual patient. Further, patient imaging data can be used to gain information on the patient bone quality and design the implant to profit from the denser bone regions. Also, the load may be tailored to the patient, based on general patient information.

Accordingly, in one example, the present system can include a system for manufacturing an implant including: at least one processor; and at least one memory storing instructions that, when executed by the at least one processor, cause the at least one processor to perform actions comprising: receiving, at a computing device, patient specific data related to a bone; generating an implant geometry of an implant based on the patient specific data; generating an initial screw location, length, and trajectory for each of a plurality of peripheral screws for the implant and a central screw for the implant; determining, at the computing device, based on the initial screw locations, lengths, and trajectories, an initial centroid of the plurality of peripheral screws and the central screw, and an initial stiffness ratio based on an initial stiffness of the plurality of peripheral screws and the central screw about a horizontal axis and an initial stiffness of the plurality of screws and the central screw about a vertical axis; and determining, by the computing device, an optimal screw location, length, and trajectory for each of the plurality of peripheral screws and the central screw for the implant using finite element analysis and a machine learning model where the optimal screw location, length, and trajectory are chosen such that a final centroid of the plurality of peripheral screw locations and the central screw location provides for one or more of a reduced or otherwise altered stiffness ratio from the initial stiffness ratio, optimal micromotion, and a reduced moment arm.

In further embodiments, the system can generate a plurality of designs for the implant, each of the plurality of designs including a different permutation of screw trajectory, length, and location for each of the plurality of peripheral screws for the implant and the central screw.

The system can help determine the optimal screw trajectory, length, and location for each of the plurality of peripheral screws and the central screw. For example, the system can include changing a portion of the implant data representing a location of the central screw from a first location to a second location; and simulating a stiffness of the implant in the horizontal axis and the vertical axis and determining a stiffness ratio. In some examples, the reduced stiffness ratio is at most a 2:1 ratio of the stiffness of the plurality of screws in the horizontal compared to the stiffness of the plurality of screws in the vertical axis. In other examples, the reduced stiffness ratio is at most a 3:1, or at most 4:1. In an example, the stiffness is equal in both axis. The stiffness ratio can also be patient specific base on patient bone anatomy and material property. The desired stiffness ratio can for each patient can be determined using a ML model based on retrospective cases, FEA evaluation and clinical outcomes.

Accordingly, in one example, the present system provides a method and system for manufacturing an implant that can include receiving, at a computing device, patient specific data related to a bone; generating an implant geometry of an implant based on the patient specific data; generating an initial screw trajectory, location, and length for each of a plurality of peripheral screws for the implant and a central screw for the implant; determining, at the computing device, based on the initial screw trajectories, locations and length, an initial centroid of the plurality of peripheral screws and the central screw, and an initial stiffness ratio based on an initial stiffness of the plurality of peripheral screws and the central screw about a horizontal axis and an initial stiffness of the plurality of screws and the central screw about a vertical axis; and determining, by the computing device, an optimal screw trajectory, location, and length for each of the plurality of peripheral screws and the central screw for the implant using material mapping, finite element analysis, and a machine learning model where the optimal screw trajectories and locations are chosen such that a final centroid of the plurality of peripheral screw locations and the central screw location provides for an optimized stiffness ratio which will reduce bone-implant micromotion, improve loading distribution, reduce strain shielding, and reduce probability of implant or bone fracture.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A method for manufacturing an implant, the method comprising:
receiving, at a computing device, patient specific data related to a bone;
generating an implant geometry of an implant based on the patient specific data;
generating an initial screw trajectory and location for each of a plurality of peripheral screws for the implant and a central screw for the implant;
determining, at the computing device, based on the initial screw trajectories and locations, an initial centroid of the plurality of peripheral screws and the central screw, and an initial stiffness ratio based on an initial stiffness of the plurality of peripheral screws and the central screw about a horizontal axis and an initial stiffness of the plurality of screws and the central screw about a vertical axis; and
determining, by the computing device, an optimal screw trajectory and location for each of the plurality of peripheral screws and the central screw for the implant using finite element analysis and a machine learning model where the optimal screw trajectories and locations are chosen such that a final centroid of the plurality of peripheral screw locations and the central screw location provides for an altered stiffness ratio from the initial stiffness ratio.

2. The method of claim 1, wherein determining the optimal screw trajectory and location for each of the plurality of peripheral screws and the central screw comprises:
changing a portion of the implant data representing a location of the central screw from a first location to a second location; and
simulating a stiffness of the implant in the horizontal axis and the vertical axis and determining a horizontal: vertical stiffness ratio.

3. The method of claim 2, wherein the altered stiffness ratio includes a reduced stiffness ratio and is at most a 2:1 ratio.

4. The method of claim 3, wherein the altered stiffness ratio is chosen to be patient specific.

5. The method of claim 1, wherein determining the optimal screw trajectory and location for each of the plurality of peripheral screws and the central screw for the implant using material mapping, finite element analysis and the machine learning model includes determining a need to offset the central screw.

6. The method of claim 1, further including determining an initial loading moment of a glenosphere on the implant, and determining an optimal screw trajectory and location for each of the plurality of peripheral screws and the central screw for the implant using finite element analysis and the machine learning model includes determining a reduced final loading moment arm.

7. The method of claim 6, further including simulating moving a glenosphere taper hole more distally and offsetting the central screw relative to the glenosphere taper of the implant to reduce the loading moment and micromotion.

8. The method of claim 1, wherein determining the optimal screw trajectory and location for each of the plurality of peripheral screws and the central screw for the implant using material mapping, finite element analysis and the machine learning model includes simulating a plurality of different screw locations and their associated stiffness centroid.

9. The method of claim 1, wherein receiving the patient specific data comprises:
receiving a scan of a patient's anatomy; and
extracting design constraints for the implant from the scan, wherein the patient specific data is from a CT scan.

10. The method of claim 1, further including generating an initial screw length for each of the plurality of peripheral screws and the central screw, and determining, by the computing device, an optimal screw trajectory, length, and location for each of the plurality of peripheral screws and the central screw for the implant using material mapping, finite element analysis and the machine learning model.

11. The method of claim 10, further including receiving, at the computing device, a bone material density distribution of the bone, wherein determining the optimal screw trajectory, length, and location for each of the plurality of peripheral screws and the central screw for the implant using finite element analysis and the machine learning model includes taking into account the bone material distribution.

12. The method of claim 11, where determining the optimal screw trajectory, length, and location for each of the plurality of peripheral screws and the central screw includes accounting for surgeon variables in the finite element analysis and the machine learning model and includes taking into account the bone material distribution.

13. The method of claim 12, wherein one or more of the surgeon variables include glenosphere size, glenosphere orientation, screw torque, screw lengths, and inclination.

14. The method of claim 1, wherein the finite element analysis and machine learning model includes generating a plurality of designs for the implant, each of the plurality of designs including a different permutation of design parameters including screw trajectory and location for each of a plurality of peripheral screws for the implant and the central screw for the implant.

15. The method of claim 1, further including using all inputs and outputs of the method to build further ML models which can include patient long term outcomes.

16. A system for manufacturing an implant, the system comprising:
at least one processor; and
at least one memory storing instructions that, when executed by the at least one processor, cause the at least one processor to perform actions comprising:
receiving, at a computing device, patient specific data related to a bone;
generating an implant geometry of an implant based on the patient specific data;
generating an initial screw trajectory and location for each of a plurality of peripheral screws for the implant and a central screw for the implant;
determining, at the computing device, based on the initial screw trajectories and locations, an initial centroid of the plurality of peripheral screws and the central screw, and an initial stiffness ratio based on an initial stiffness of the plurality of peripheral screws and the central screw about a horizontal axis and an initial stiffness of the plurality of screws and the central screw about a vertical axis; and
determining, by the computing device, an optimal screw trajectory and location for each of the plurality of peripheral screws and the central screw for the implant using finite element analysis and a machine learning model where the optimal screw trajectories and locations are chosen such that a final centroid of the plurality of peripheral screw locations and the central screw location provides for an altered stiffness ratio from the initial stiffness ratio.

17. The system of claim 16, further comprising generating a plurality of designs for the implant, each of the plurality of designs including a different permutation of screw trajectory and location for each of the plurality of peripheral screws for the implant and the central screw.

18. The system of claim 17, wherein determining the optimal screw trajectory and location for each of the plurality of peripheral screws and the central screw comprises:
changing a portion of the implant data representing a location of the central screw from a first location to a second location; and
simulating a stiffness of the implant in the horizontal axis and the vertical axis and determining a stiffness ratio.

19. The system of claim 18, wherein the altered stiffness ratio is determined based on a specific patient need.

20. A glenoid baseplate comprising:
a baseplate body formed according to a PMI (patient-matched implant); and
the baseplate body including a plurality of mounting holes extending though the baseplate body including two or more peripheral holes and a central hole;
wherein locations and trajectories of the plurality of mounting holes on the baseplate is chosen according to a method comprising:
generating an initial screw trajectory and location for each of a plurality of peripheral screws for the implant and a central screw for the implant;
determining, at a computing device, based on the initial screw trajectories and locations, an initial centroid of the plurality of peripheral screws and the central screw, and an initial stiffness ratio based on an initial stiffness of the plurality of peripheral screws and the central screw in a horizontal axis and an initial stiffness of the plurality of screws and the central screw in a vertical axis; and
determining, by the computing device, an optimal screw trajectory and location for each of the plurality of peripheral screws and the central screw for the implant using finite element analysis and a machine learning model where the optimal screw trajectories and locations are chosen such that a final centroid of the plurality of peripheral screw locations and the central screw location provides for a reduced stiffness ratio from the initial stiffness ratio.
